(19) United States
(12) Patent Application Publication
(10) Pub. No.: US 2025/0375554 A1
(43) Pub. Date: Dec. 11, 2025

(54) FUNCTIONALIZED POLYURETHANE AND MENISCUS PROSTHESIS, THE PREPARATION METHOD THEREOF AND THE USES THEREFOR

(71) Applicant: WEST CHINA HOSPITAL, SICHUAN UNIVERSITY, Chengdu (CN)

(72) Inventors: Weili FU, Chengdu (CN); Xia JIANG, Chengdu (CN); Yi ZHOU, Chengdu (CN)

(21) Appl. No.: 19/307,115

(22) Filed: Aug. 22, 2025

(30) Foreign Application Priority Data
Aug. 26, 2024 (CN) 202411177632.5

(51) Int. Cl. A61L 27/44; A61F 2/30; A61L 27/18; C08B 37/08; C08G 18/10; C08G 18/32; C08G 18/38; C08J 7/04

(52) U.S. Cl. CPC A61L 27/443; A61F 2/30756; A61F 2/3094; A61L 27/18; C08B 37/0072; C08G 18/10; C08G 18/3206; C08G 18/3819; C08J 7/0427; A61L 2430/24; C08J 2375/08; C08J 2405/00

(57) ABSTRACT

A functionalized polyurethane, meniscus prosthesis, the preparation method thereof, and the uses therefor are disclosed. The meniscus prosthesis is manufactured by using polyurethane as the base material and incorporating a self-healing chemical bond, the dynamic dimethylglyoxime-urethane group. Additionally, hyaluronic acid coated with adamantane structures is introduced onto the surface of the self-healing polyurethane elastomer, creating a supramolecular lubricating coating with stress response characteristics. This successfully mimics the dynamic self-healing ability and physiological cyclic lubrication mechanism of natural meniscus, offering a novel solution for the treatment of meniscus injuries and holding great potential value in clinical applications.

### FIELD OF THE INVENTION

The present invention belongs to the field of biomedical material technology, and specifically relates to a functionalized polyurethane, meniscus prosthesis, the preparation method therefor, and the uses thereof.

### BACKGROUND OF THE INVENTION

The precise anatomical structure of the meniscus is crucial for the complex movement and stable support of the knee joint. Clinically, for irreparable meniscus injuries and severe meniscus degeneration, total meniscectomy remains the preferred treatment method; however, this inevitably leads to instability of the knee joint, wear and tear of articular cartilage, and ultimately the formation of osteoarthritis. To address this issue, a series of meniscus substitutes have been developed. However, the geometric characteristics of most meniscus replacements do not match well with those of natural menisci, making it impossible to restore the initial stress distribution within the joint compartment. Secondly, although most constructed tissue-engineered meniscus materials have a compressive modulus similar to that of natural menisci, their tensile modulus differs significantly from the circumferential/radial tensile modulus of natural menisci, making them incompatible with the anisotropic mechanical characteristics of natural menisci. When subjected to load, significant deformation occurs due to circumferential expansion, leading to local collapse and fracture of the meniscus scaffold. In addition, the repeated contact and friction between the femur and tibia on the meniscus also lead to severe wear of the tissue-engineered meniscus. Due to the abundance of joint fluid and proteases in the knee joint cavity, the rapid degradation of meniscus materials after implantation is also one of the current drawbacks.

Polyurethane elastomers are a type of polymer material with unique properties. They combine the elasticity of rubber with the durability of plastics, exhibiting excellent tensile strength, tear resistance, and impact resistance. These characteristics make them perform well under heavy pressure and impact, especially in high-friction environments where they maintain good performance. Furthermore, due to the excellent biocompatibility of polyurethane, it has been applied in the medical field, such as in the manufacture of artificial blood vessels, cardiac pacemakers, and artificial bones.

Self-healing materials are a novel type of smart materials capable of repairing their mechanical damage and restoring their original functions under certain external stimuli. They rely on reversible covalent or non-covalent bonding forces and other reversible interactions, achieving complete performance recovery through reversible breaking and formation under external stimuli. Theoretically, they can undergo an unlimited number of repairs.

The lubricating effect of articular cartilage and meniscus is crucial in knee joint movement. Hyaluronic acid and lubricin in joint synovial fluid are the main lubricating components. The knee joint bears extremely complex and demanding loads during daily activities. Even under high loads, the friction coefficient of articular cartilage in the knee joint during movement is as low as 10-3. Based on the physiological lubrication phenomenon of the knee joint, tissue-engineered meniscus/cartilage scaffolds with self-lubricating properties have become one of the current research hotspots, thereby increasing material lifespan and delaying the progression of osteoarthritis.

Currently, meniscus substitutes face challenges such as poor mechanical properties, severe wear, susceptibility to degradation, short service life, and inadequate compatibility with natural meniscus. Therefore, there is an urgent need to develop a novel meniscus prosthesis that mimics the dynamic self-repair capability and physiological cyclic lubrication mechanism of the natural meniscus, providing a novel solution for the treatment of meniscus injuries.

### Content of the Invention

In order to address the issues of the prior art, the present invention provides a functionalized polyurethane, meniscus prosthesis, as well as the preparation method therefor and the uses thereof.

A functionalized polyurethane, characterized in that it comprises a self-healing polyurethane elastomer and an adamantane-hyaluronic acid composite, wherein the adamantane-hyaluronic acid composite is coated on the surface of the self-healing polyurethane elastomer, and the thickness of the adamantane-hyaluronic acid composite is 10-20 μm;
the self-healing polyurethane elastomer is made from raw materials in the following parts by weight:
1-2 parts of hard segment raw material,
4.5-9 parts of soft segment raw material,
0.262-0.522 parts of dimethylglyoxime,
0.412-0.824 parts of chain extender,
0.04-0.08 parts of crosslinking agent;
the adamantane-hyaluronic acid composite is made from raw materials in the following parts by weight:
10-20 parts of hyaluronic acid,
2.3-4.6 parts of diepoxy compound,
0.5-5 parts of adamantane.

Preferably, the self-healing polyurethane elastomer is made from raw materials in the following parts by weight:
1 part of hard segment raw material,
4.5 parts of soft segment raw material,
0.262-0.522 parts of dimethylglyoxime,
0.412 parts of chain extender,
0.04 parts of crosslinking agent.

Preferably, the self-healing polyurethane elastomer is made from raw materials in the following parts by weight:
1 part of hard segment raw material,
4.5 parts of soft segment raw material,
0.392 parts of dimethylglyoxime,
0.412 parts of chain extender,
0.04 parts of crosslinking agent.

Preferably, the adamantane-hyaluronic acid composite is made from raw materials in the following parts by weight:
10 parts of hyaluronic acid,
2.3 parts of diepoxy compound,
0.5-5 parts of adamantane.

Preferably, the adamantane-hyaluronic acid composite is made from raw materials in the following parts by weight:
10 parts of hyaluronic acid,
2.3 parts of diepoxy compound,
5 parts of adamantane.

Preferably, the raw material for the hard segment is selected from at least one of isophorone diisocyanate and polycaprolactone triol;
and/or, the raw material for the soft segment is selected from at least one of polytetramethylene ether glycol (PTMEG) and polypropylene oxide;
and/or, the chain extender is selected from at least one of 1,4-butanediol and glycerol;
and/or, the crosslinking agent is selected from at least one of aminocyclodextrin and ethylenediamine;
and/or, the diepoxy compound is selected from at least one of 1,4-butanediol diglycidyl ether (BDDE) and 1,6-hexanediol diglycidyl ether.

The present invention also provides a preparation method for above functionalized polyurethane, comprising the following steps:
Step 1: the hard segment raw material reacts with the soft segment raw material to obtain mixture A;
Step 2: mixture A reacts with dimethylglyoxime to obtain mixture B;
Step 3: mixture B reacts with chain extender and crosslinking agent, to obtain self-healing polyurethane elastomer after purification;
Step 4: hyaluronic acid reacts with diepoxide compound to obtain mixture C;
Step 5: mixture C reacts with adamantane to obtain adamantane-hyaluronic acid complex;
Step 6: the adamantane-hyaluronic acid composite is uniformly applied onto the surface of the self-healing polyurethane elastomer to obtain self-lubricating and self-healing polyurethane elastomer materials.

Preferably, in step 1, the reaction temperature is 60-80° C., and the reaction time is 45 min to 1.5 h;
and/or, in step 2, the reaction temperature is 60-80° C., and the reaction time is 1-2 h;
and/or, in step 3, the reaction temperature is 60-80° C., and the reaction time is 0.5-2 h;
and/or, in step 4, the reaction temperature is 20-50° C., and the reaction time is 6-12 h;
and/or, in step 5, the reaction temperature is 40-80° C., and the reaction time is 12-36 h.

The present invention also provides the use of above functionalized polyurethane in the preparation of meniscus prostheses.

The present invention also provides a meniscus prosthesis, which is made of above functionalized polyurethane.

The present invention designs a polyurethane meniscus prosthesis with self-lubricating and self-healing functions. The meniscus prosthesis is made of polyurethane as the base material, introducing a self-healing chemical bond—a dynamic dimethylglyoxime-urethane group. Then, an adamantane structure is introduced onto the surface of the self-healing polyurethane elastomer, carrying hyaluronic acid. That possesses high modulus, high elasticity, and high lubrication characteristics, while exhibiting good biocompatibility and cartilage protection effects, providing a new solution for the treatment of meniscus injuries.

Obviously, based on the above content of the present invention, according to the common technical knowledge and the conventional means in the field, other various modifications, alternations, or changes can further be made, without department from the above basic technical spirits.

With reference to the following specific examples, the above content of the present invention is further illustrated. But it should not be construed that the scope of the above subject matter of the present invention is limited to the following examples. The techniques realized based on the above content of the present invention are all within the scope of the present invention.

### DESCRIPTION OF FIGURES

FIG. 1. Diagram of the self-healing test for the self-healing polyurethane meniscus body material.

FIG. 2. The results for the tensile property of polyurethane meniscus materials. Panel a represents the self-healing efficiency of the self-healing polyurethane elastomer; panel b represents the tensile modulus of the self-healing polyurethane elastomer; panel c represents the ultimate tensile strength of the self-healing polyurethane elastomer; and panel d represents the tensile stress of DPU2-CD.

FIG. 3. The results for the compression performance of polyurethane meniscus materials. Panel a represents the compressive modulus of the self-healing polyurethane elastomer, and panel b denotes the compressive stress of DPU2-CD.

FIG. 4. The test results for friction coefficient of polyurethane meniscus.

FIG. 5. The test results for the biocompatibility of polyurethane meniscus. Panels a and f represent the quantitative graphs of cell viability evaluation and results for different material groups using the live-dead cell staining; panels b, e, and g represent the evaluation of the impact of different material groups on chondrocyte proliferation using EDU labeling and CCK-8 method; and panels c and d represent the evaluation of cell migration ability of different material groups using scratch assay and Transwell chamber assay.

FIG. 6. Evaluating the impact of materials on the expression of anabolic and catabolic proteins in chondrocytes using immunofluorescence technology. Panels a and d show the expression and quantification results of the anabolic protein COL-2; panels b and c display the expression and quantification results of the catabolic protein MMP-13.

FIG. 7. Polyurethane meniscus prosthesis implanted in a rabbit.

FIG. 8. Gross observations and Micro CT scanning assessments on the femoral condyles, femoral trochlea, and knee joint cartilage of tibial plateau for three groups of rabbits at 6 and 12 weeks post-surgery, to quantitatively and qualitatively analyze the protective effect on knee joint cartilage. Panel a shows the gross images of the femoral condyles of the three groups at 6 and 12 weeks post-surgery; panel b shows the gross images of the femoral trochlea of the three groups at 6 and 12 weeks post-surgery; panel c shows the gross images of the tibial plateau of the three groups at 6 and 12 weeks post-surgery; panel d shows the Micro CT images of the three groups at 6 weeks post-surgery; panel e shows the Micro CT images of the three groups at 12 weeks post-surgery; panel f shows the gross score graphs of the cartilage at 6 and 12 weeks post-surgery; panel g shows the quantified assessment graphs of the osteophyte volume of the knee joints in the three groups at 6 and 12 weeks post-surgery; and panel h shows the quantified assessment graphs of the subchondral bone density of the knee joint cartilage in the three groups at 6 and 12 weeks post-surgery.

FIG. 9. The femtosecond laser detection result images of articular cartilage. Panel a represents the collagen and metabolite assessment images of the subtotal resection group at 6 and 12 weeks after surgery; panel b represents the collagen and metabolite assessment images of the PU group at 6 and 12 weeks after surgery; panel c represents the collagen and metabolite assessment images of the DPU@HA group at 6 and 12 weeks after surgery; and panel d represents the collagen and metabolite assessment images of natural articular cartilage at 6 and 12 weeks after surgery.

### EXAMPLES

In the following examples and experimental examples, reagents and raw materials not specifically mentioned are commercially available products.

### Example 1: Preparation of Self-Lubricating and Self-Healing Polyurethane Elastomer Meniscus Prosthesis

This example provided a self-lubricating and self-healing polyurethane elastomer meniscus prosthesis, and its preparation method was as follows:

### 1. Synthesis of Self-Healing Polyurethane Elastomer (DPU-CD)

Under nitrogen protection, first, 5 g of isophorone diisocyanate (IPDI, Aladdin, CAS 4098-71-9) was added to a three-neck flask containing 15 ml of toluene. Subsequently, 22.5 g of polytetramethylene ether glycol (PTMG, Aladdin, CAS 25190-06-1) was added, with a molar ratio of 1:1 to IPDI, and the mixture was heated and stirred at 60° C. for 60 min to form the base prepolymer. Then, according to different self-healing performance requirements, the following amounts of dimethylglyoxime (DMG) were slowly added:
DPU1: Adding 1.31 g of DMG, with a molar ratio of 1:1 to IPDI, to introduce self-healing properties to a moderate extent;
DPU2: Adding 1.96 g of DMG, with a molar ratio of 1.5:1 to IPDI, to introduce self-healing properties to a higher extent;
DPU3: Adding 2.61 g of DMG, with a molar ratio of 2:1 to IPDI, to introduce self-healing properties to the greatest extent.

In each case, then, 2.06 g of 1,4-butanediol (BDO, Aladdin, CAS 110-63-4) was slowly added, followed by further reacting at 60° C. for 60 min, to carry out the chain extension reaction, forming a polyurethane prepolymer. Afterwards, 0.2 g of aminocyclodextrin (CD, Aladdin, CAS 29390-67-8) was added, and continue to react at 60° C. for 2 h. After completion of the reaction, the mixture was dissolved in acetone at room temperature and then precipitated in cyclohexane to obtain white solid precipitates. The precipitates were filtered, washed with cyclohexane, and finally dried under reduced pressure at room temperature for 5 h to obtain white powders of self-healing polyurethane elastomers DPU1-CD, DPU2-CD, and DPU3-CD.

### 2. Preparation of Adamantane-Hyaluronic Acid Complex (AD-HA)

Firstly, 10 g of hyaluronic acid (HA) was reacted with 2.3 g of 1,4-butanediol diglycidyl ether (as a representative of diepoxy compounds) at 50° C. The reaction lasted for 12 h, ensuring uniform mixing. After the reaction was complete, unreacted 1,4-butanediol diglycidyl ether and by-products were removed through dialysis, yielding an epoxy modified HA solution. Subsequently, the solution containing the epoxy modified HA derivative was pre-frozen at −20° C. for approximately 2 h. Then, it was placed in a freeze dryer and dried once at a low pressure of 0.1 mbar, with the temperature controlled at around −20° C. for 8 h to promote the sublimation of ice crystals. Then, a secondary drying process was carried out, continuing the drying at a pressure of 0.01 mbar and a temperature of 20° C. for 4 h to remove residual moisture, ultimately yielding epoxy modified HA derivatives as loose and porous solid.

Then, the resultant epoxy-modified HA derivative reacted with adamantane at 80° C. for 24 h, to prepare adamantine-hyaluronic acid (AD-HA) complex. The amount of adamantine was determined based on its molar ratio with the epoxy group in epoxy-modified HA, and this ratio could generally be set at 1:1 to 1:10. If using an equivalent amount of adamantane to the epoxy-modified HA derivative at a molar ratio of 1:1, 5 g of adamantane was used; if at a molar ratio of 1:10, 0.5 g of adamantane was used to adjust the lubrication performance and mechanical strength of the complex. Finally, by filtration and washing, a pure adamantane-hyaluronic acid composite (AD-HA) was obtained.

### 3. Synthesis of Self-Lubricating and Self-Repairing Polyurethane Elastomer Meniscus Material (DPU-CD+AD-HA)

First, 20 g of self-healing polyurethane elastomer powder (DPU-CD) was accurately weighed and dissolved in 50 mL of acetone. The solution was thoroughly stirred using a magnetic stirrer at room temperature (approximately 25° C.), until the powder was completely dissolved, forming a uniform solution. Then, the solution was poured into a pre-cleaned and prepared flat polyester film mold, using a scraper to ensure that the solution evenly covered the entire mold surface, avoiding the formation of bubbles or uneven thickness. Then, the mold containing the polyurethane solution was placed into an oven preheated to 40° C. The drying process lasted for about 3 h, until acetone was completely evaporated, forming a uniform and continuous polyurethane film.

Subsequently, a lubricating coating of adamantane-hyaluronic acid composite (AD-HA) was prepared. 5 g of composite powder was dispersed in 10 ml of deionized water, and subjected to an ultrasonic bath for 10 min to form a uniform coating solution. Then, a soft brush or scraper was used to evenly apply the coating onto the surface of the formed polyurethane film, ensuring the uniformity and consistency of the coating. The coating thickness was controlled within the range of 10-20 μm, with a specific coating thickness of 15 μm in this example.

### 4. Preparation of Self-Lubricating and Self-Repairing Polyurethane Elastomer Meniscus Prosthesis

After the coating process, CT images and CAD software were utilized to perform a three-dimensional reconstruction of the actual meniscus, thereby obtaining its precise geometric shape and dimensional information. Based on this, a digital model of the meniscus prosthesis was designed. Subsequently, the corresponding mold was fabricated using high-precision 3D printing technology known as Stereolithography (SLA). Photocurable resin was chosen as the 3D printing material to ensure the accuracy and complexity of the mold. The print nozzle diameter was set to 50 μm, and the print platform temperature was maintained at 25° C. Finally, using thermoplastic polyurethane (TPU) material, a meniscus prosthesis with self-healing and self-lubricating properties was constructed by an inverse molding process. The TPU particles were melted at 180° C., poured into a 3D printed mold, applied with a pressure of 50 Pa, and maintained at this temperature for 2 min to ensure uniform material filling of the mold. After removing the molded meniscus prosthesis, post-processing was performed, including curing at room temperature for 24 hours to improve mechanical properties, and surface treatment using fine sandpaper to achieve the desired smoothness.

### Example 2: Self-Lubricating and Self-Healing Polyurethane Elastomer Meniscus Prosthesis 2

This sample was prepared from 10 g of isophorone diisocyanate, 22.5 g of polytetramethylene ether glycol (PTMG), 1.31 g of dimethylglyoxime, 4.12 g of 1,4-butanediol, 0.2 g of aminocyclodextrin, 20 g of hyaluronic acid, 2.3 g of 1,4-butanediol diglycidyl ether, and 5 g of adamantane. Except for the different proportions of raw materials, the preparation method was the same as in Example 1.

### Example 3: Self-Lubricating and Self-Healing Polyurethane Elastomer Meniscus Prosthesis 3

This sample was prepared from 5 g of isophorone diisocyanate, 45 g of polytetramethylene ether glycol (PTMG), 2.61 g of dimethylglyoxime, 2.06 g of 1,4-butanediol, 0.4 g of aminocyclodextrin, 10 g of hyaluronic acid, 4.6 g of 1,4-butanediol diglycidyl ether, and 0.5 g of adamantane. Except for the different proportions of raw materials, the preparation method was the same as in Example 1.

### Comparative Example 1

This comparative example provided the control samples used in the experiment:

### 1. PU

Under nitrogen protection, first, 5 g of isophorone diisocyanate (IPDI, Aladdin, CAS 4098-71-9) was added to a three-neck flask containing 15 ml of toluene. Subsequently, 22.55 g of polytetramethylene ether glycol (PTMG, Aladdin, CAS 25190-06-1) was added, with a molar ratio of 1:1 to IPDI, and the mixture was heated and stirred at 60° C. for 60 min to form the base prepolymer. Then, 2.06 g of 1,4-butanediol (BDO, Aladdin, CAS 110-63-4) was slowly added, followed by further reacting at 60° C. for 60 min, to carry out the chain extension reaction, forming a polyurethane prepolymer. After completion of the reaction, the mixture was dissolved in acetone at room temperature and then precipitated in cyclohexane to obtain white solid precipitates. The precipitates were filtered, washed with cyclohexane, and finally dried under reduced pressure at room temperature for 5 h to obtain pure polyurethane (PU) as white powders.

### 2. Self-Healing Polyurethane Elastomer (DPU-CD)

The sample was prepared from isophorone diisocyanate, PTMG, dimethylglyoxime, 1,4-butanediol, and aminocyclodextrin, and the preparation method and the amount ratio of raw materials were the same as those in step 1 of Example 1.

The technical solution of the present invention would be further illustrated by following experiments. The samples used in the experiments were prepared according to the method described in Example 1.

### Experimental Example 1: Performance Testing of Self-Lubricating and Self-Healing Polyurethane Meniscus Material

The DPU-CD+AD-HA, DPU-CD, and PU samples used in this experimental example were all prepared according to the method described in Example 1 or Comparative example 1.

### 1. Experimental Method

Self-healing test: After synthesizing the self-healing polyurethane (DPU-CD), the DPU-CD was evenly divided into two parts using a cutter, and the two parts were colored red and blue respectively. Under room temperature drying at 37° C., the two parts were brought into contact with each other. After 2 minutes, the two parts were pulled to determine whether they could heal themselves.

Mechanical property testing: The mechanical properties of polyurethane materials with different compositions were tested using an electronic universal testing machine (Instron, USA). To characterize the tensile properties of polyurethanes with different compositions, rectangular specimens with a length of 40 mm, a width of 5 mm, and a thickness of 0.8 mm were constructed and subjected to tensile testing (tensile rate: 50 mm/min). To characterize the compressive properties of polyurethane materials, cylindrical polyurethane structures with a diameter of 10 mm and a thickness of 2.5 mm were constructed and subjected to compressive testing (compression rate: 10 mm/min). The cyclic tensile test was set with a deformation of 10% and 50 cycles, while the cyclic compression test was set with a deformation of 20% and 50 cycles.

Friction performance test: After selecting the optimal DPU-CD based on the previous mechanical property, a supramolecular coating was added to construct DPU-CD+AD-HA. Using DPU-CD and PU as control samples, the friction test of the materials was carried out using a universal reciprocating friction tester. The oscillation amplitude, frequency, applied load, and test time were set to 4 mm, 1 Hz, 1 N, and 600 s, respectively. The COF measurement began at 0.495 seconds after the start of oscillation, and data were recorded at 1-second intervals.

### 2. Experimental Results

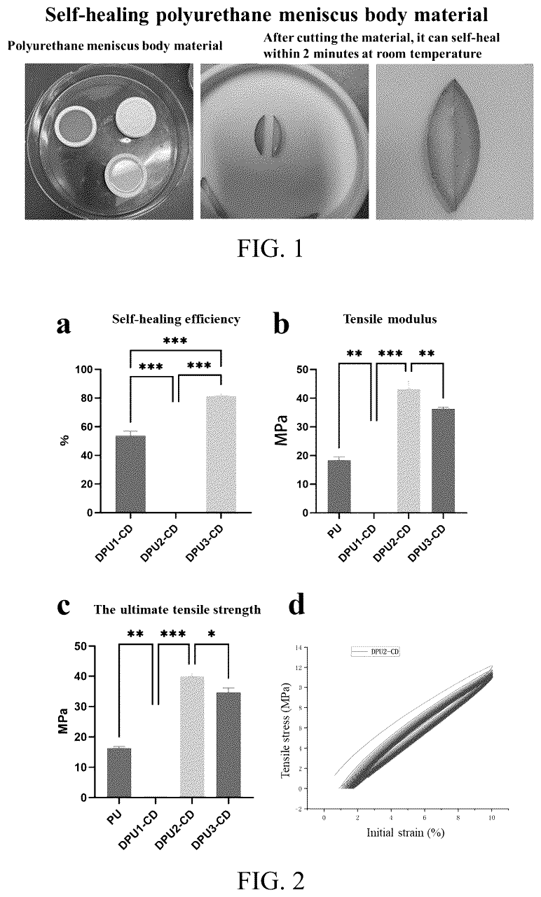

The self-healing test results are shown in FIG. 1, where DPU-CD self-healed within 2 minutes.

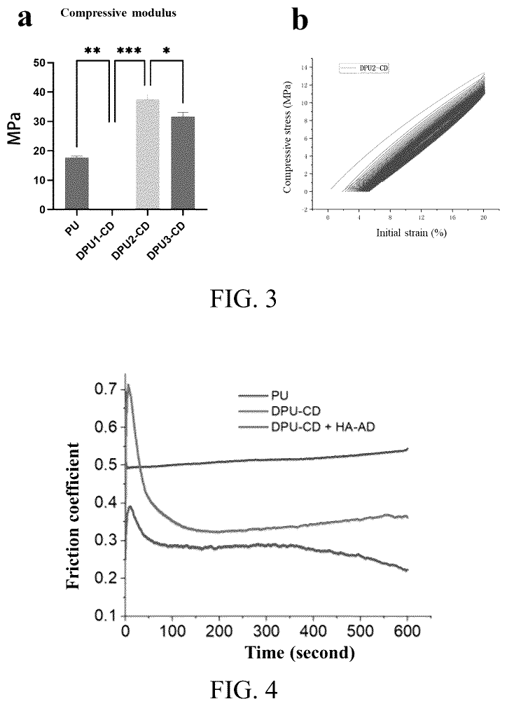

The test results of tensile performance are shown in FIG. 2 panels a-d. DPU2-CD, while exhibiting self-healing efficiency, had the highest tensile modulus compared to other groups, which exceeded the circumferential tensile modulus level of natural meniscus, indicating that the internal structure of this meniscus substitute could provide sufficient tensile performance. The test results of compression performance are shown in FIG. 3 panels a-b. DPU2-CD, while exhibiting self-healing efficiency, had the highest compression modulus compared to other groups. Therefore, DPU2-CD would be used in subsequent experiments.

The test results of friction performance are shown in FIG. 4: The DPU-CD+AD-HA group exhibited the lowest friction coefficient compared to the other groups. Over time, it remained stable at a friction coefficient of less than 0.3, whereas the friction coefficient of the PU group was greater than 0.5.

The experimental results above indicated that the self-lubricating and self-healing polyurethane meniscus material constructed in the present invention exhibited excellent healing capabilities. Among various ratios of dimethylglyoxime, DPU2-CD demonstrated superior mechanical properties while maintaining self-healing efficiency, and DPU-CD+AD-HA demonstrated good self-lubricating properties.

### Experimental Example 2: In Vitro Investigation of the Biocompatibility of Self-Lubricating and Self-Healing Polyurethane Meniscus Material

In this experimental example, the following groups were included: the ordinary PU group, which only contained pure PU material; the DPU group, which contained DPU-CD material; the DPU@HA group, which contained DPU-CD+AD-HA material; all the materials mentioned above were prepared according to the method described in Example 1 or Comparative example 1.

### 1. Experimental Method

After screening out the optimal ratio of materials in the aforementioned materials section, chondrocytes were used as seed cells and loaded onto different polyurethane materials. The group in the well plates served as a control. After three-dimensional co-culture, the proliferation of cells on the scaffold materials was detected using the CCK-8 method and EDU method. After labeling with live-dead cell dyes, the activity of cells on the scaffold was observed using a laser confocal microscope. Rhodamine-labeled phalloidin was used for cytoskeleton staining to observe the morphology of cells on the scaffold. Immunofluorescence was used to detect cartilage synthesis and catabolismand migration and scratch experiments were performed to test cell migration ability.

### 2. Experimental Results

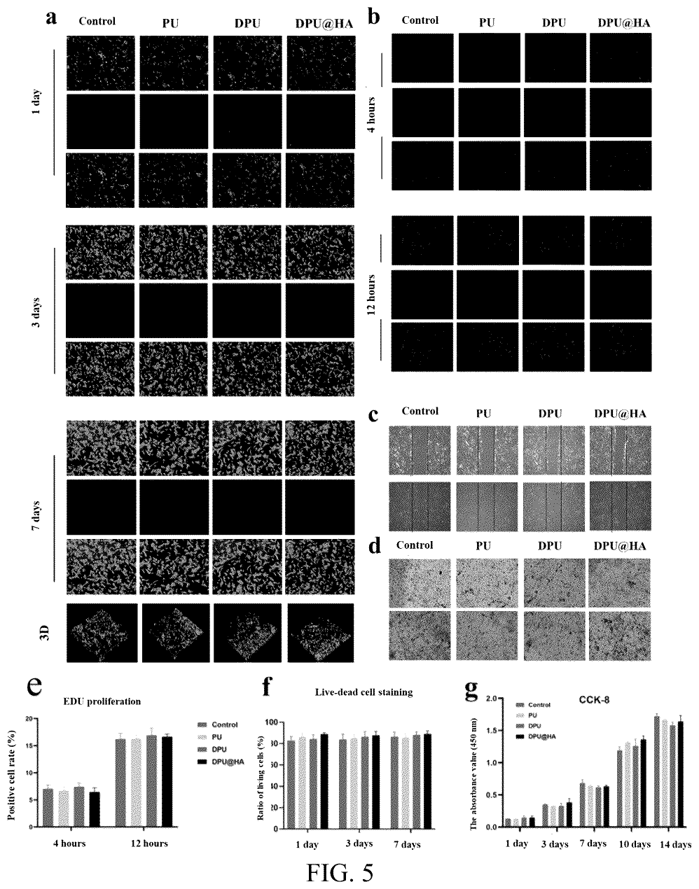

After carrying out the chondrocyte co-culture experiment, we evaluated the cell viability of different material groups using the live-dead staining method. The results are shown in FIG. 5 panels a and f. On the 1st, 3rd, and 7th days of co-culture, the number of viable cells in the PU group, DPU group, and DPU@HA group did not show statistically significant differences compared to the control group. This finding indicated that none of the three tested materials exhibited significant cytotoxicity towards chondrocytes. Furthermore, to evaluate the effects of these materials on chondrocyte proliferation, we used EDU (5-ethynyl-2′-deoxyuridine) labeling and CCK-8 (Cell Counting Kit-8) assay. The experimental results indicated that there was no significant statistical difference in the cell proliferation levels of the PU group, DPU group, and DPU@HA group compared to the control group (FIG. 5 panels b, e, and g). This suggested that these materials did not inhibit the DNA proliferation of chondrocytes. Regarding cell migration ability, FIG. 5 panels c and d showed no significant differences between the three material groups and the control group. Additionally, we evaluated the effects of the materials on the expression of anabolic and catabolic proteins in chondrocytes using immunofluorescence technology. The results indicated that there were no significant differences in the expression of anabolic protein COL-2 (type II collagen) among the three groups of materials compared to the control group (see FIG. 6 panels a and d). Similarly, no significant differences were observed in the expression of catabolic protein MMP-13 (matrix metalloproteinase-13) (see FIG. 6 panels b and c).

The above results indicated that these materials exhibited good biocompatibility with chondrocytes, neither inhibiting cell proliferation nor affecting cell migration ability, and having no negative impact on the synthesis and catabolism processes of chondrocytes.

### Experimental Example 3: In Vivo Investigation of the Effect of Self-Lubricating and Self-Healing Polyurethane Meniscus Prosthesis

In this experimental example, there are two groups: the PU group, which involved the addition of pure PU material; and the DPU@HA group, which involved the addition of DPU-CD+AD-HA material. The materials used in the above groups were all prepared according to the methods described in Example 1 or Comparative example 1.

### 1. Experimental Method

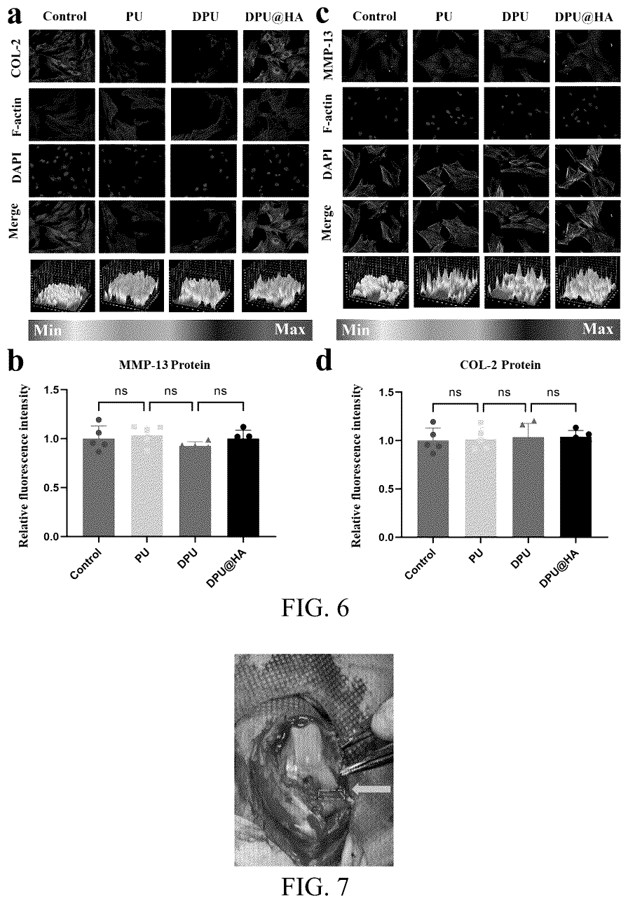

Male rabbits weighing between 2.5 and 3.0 kg were selected for experiments lasting 6 and 12 weeks, respectively. All rabbits were randomly divided into three groups: subtotal meniscectomy group (n=5 at each time point), PU-implanted material group (n=5 at each time point), and DPU@HA-implanted group (n=5 at each time point). In the implanted groups, a model of medial meniscus resection was first constructed in the rabbits, and then the PU or DPU@HA meniscus scaffolds were implanted to evaluate their in vivo applicability. The aforementioned meniscus scaffold was soaked in 75% alcohol for 30 min, and then disinfected with ultraviolet radiation for 1 h. All surgeries were performed under sterile conditions. Before surgery, the rabbits were anesthetized with sodium pentobarbital (3.5 mL/kg) via marginal ear vein injection, and fixed on the operating table. The skin preparation of both lower limbs was carried out, and then disinfected with iodophor. The skin and subcutaneous tissue were peeled off to expose the medial collateral ligament; the medial collateral ligament was incised, and then the joint capsule was opened to expose the medial meniscus; the junction between the posterior/anterior horn of the medial meniscus and the tibial plateau was incised, and the medial meniscus was completely removed; the polyurethane meniscus prosthesis constructed in the example was sutured to the ligament and joint capsule, and fixed on the tibial plateau; the joint cavity was closed, the medial collateral ligament and other anatomical tissues were reconnected or sutured with non-absorbable sutures, and then the legs were disinfected with alcohol cotton balls; finally, the rabbits were allowed to return to their cages, ensuring their free movement. For the post-surgery animals, penicillin was intramuscularly administered once a day for 3 days, to prevent infection. At 6 and 12 weeks post-surgery, all rabbits were euthanized, and their femurs, tibias, and meniscus implants were removed for gross observation and histological evaluation. FIG. 7 shows the placed polyurethane meniscus prosthesis (indicated by the yellow arrow and green dashed box).

Femtosecond laser multimodal scanning: the tibial plateau cartilage was cut into 2 mm-thick slices with a blade, placed on a stage, and immediately proceeded with machine detection. The wavelength was set to 900-1200 nm, pulse frequency to 10 Hz, pulse width to 5 fs, and the channels were selected as two-photon fluorescence 2PFE, three-photon fluorescence 3PFE, second harmonic generation SHG, and third harmonic generation THG.

### 2. Experimental Results

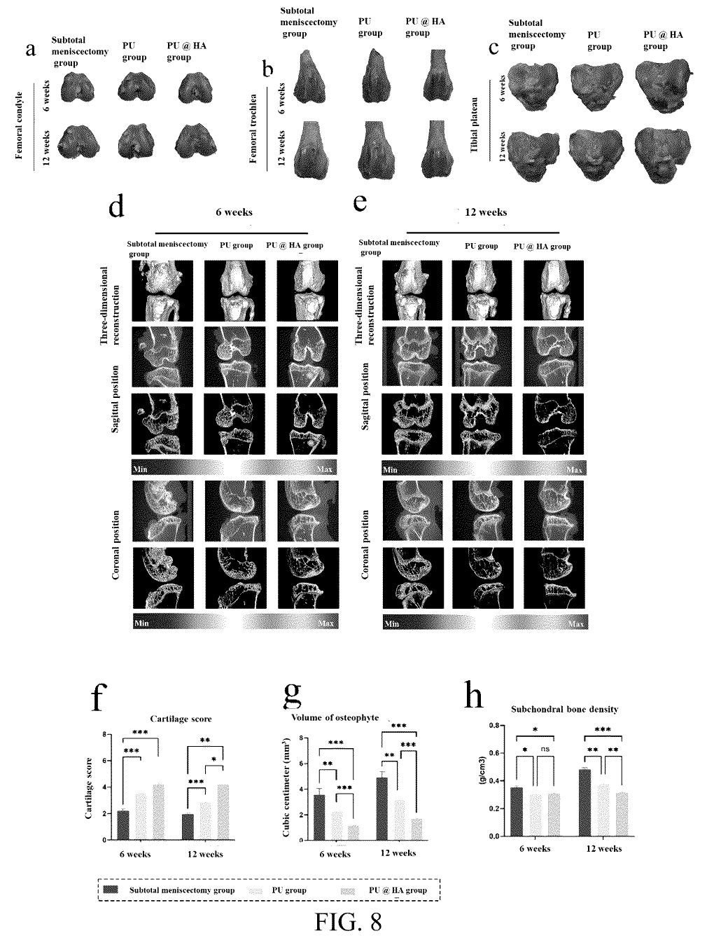

As shown in FIGS. 8, at 6 and 12 weeks post-surgery, gross observation and Micro CT scanning were carried out on the articular cartilage of the femoral condyle, femoral trochlea, and tibial plateau in three groups of rabbits, to quantitatively and qualitatively analyze the protective effect of the cartilage. As shown in FIG. 8 panels a, b, c, and f, through gross observation of the articular cartilage of the femoral condyle, femoral trochlea, and tibial plateau, the subtotal meniscectomy control group exhibited significant cartilage degeneration, including severe wear on the tibial plateau and femoral condyle cartilage, as well as cartilage damage in the femoral trochlea area. The Micro CT scanning assessment results revealed significant osteophyte proliferation and pronounced collapse of the medial compartment, accompanied by subchondral bone sclerosis. The degree of cartilage wear in the PU group ranged from moderate to severe. Although meniscus function had been restored to some extent, the cartilage protection effect was limited. In the DPU@HA group, the degree of cartilage wear was mild, no osteophyte proliferation was observed, the medial compartment remained stable, and no subchondral bone sclerosis was present (FIG. 8 panels d, e, g, and h).

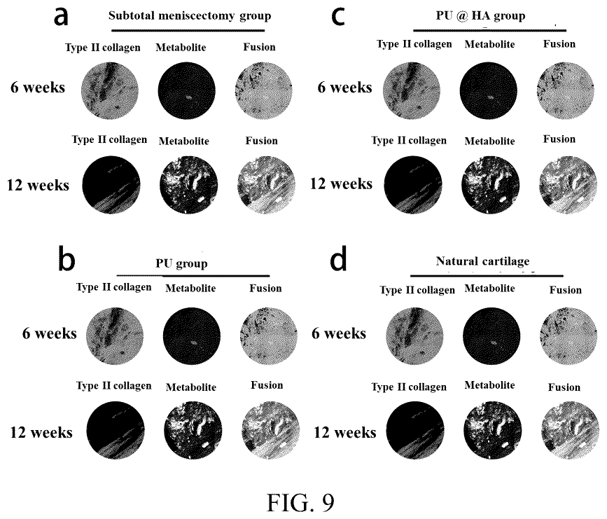

As shown in FIG. 9, in the subtotal meniscectomy group, most of the fibrous collagen in the cartilage was disordered and partially missing, with metabolic imbalance (FIG. 9 panel a). In the PU group, some fibers were arranged in disorder, and the structure was incomplete (FIG. 9 panel b). The DPU@HA group had a structure similar to that of natural articular cartilage, with stable metabolism (FIG. 9 panels c and d). The above results indicated that the self-lubricating and self-healing polyurethane meniscus prosthesis had a cartilage-protective effect.

The above experimental examples demonstrated that the self-lubricating and self-healing polyurethane meniscus prosthesis constructed in the present invention not only possessed high modulus, high elasticity, and high lubrication characteristics, but also exhibited good biocompatibility and cartilage protection effects. It achieved the imitation of dynamic self-healing and physiological cyclic lubrication of natural meniscus, and held great potential application value in constructing meniscus substitutes.

1. A functionalized polyurethane, characterized in that it comprises a self-healing polyurethane elastomer and an adamantane-hyaluronic acid composite, wherein the adamantane-hyaluronic acid composite is coated on the surface of the self-healing polyurethane elastomer, and the thickness of the adamantane-hyaluronic acid composite is 10-20 μm;
the self-healing polyurethane elastomer is made from raw materials in the following parts by weight:
1-2 parts of hard segment raw material,
4.5-9 parts of soft segment raw material,
0.262-0.522 parts of dimethylglyoxime,
0.412-0.824 parts of chain extender,
0.04-0.08 parts of crosslinking agent;
the adamantane-hyaluronic acid composite is made from raw materials in the following parts by weight:
10-20 parts of hyaluronic acid,
2.3-4.6 parts of diepoxy compound,
0.5-5 parts of adamantane.

2. The functionalized polyurethane according to claim 1, characterized in that:
the self-healing polyurethane elastomer is made from raw materials in the following parts by weight:
1 part of hard segment raw material,
4.5 parts of soft segment raw material,
0.262-0.522 parts of dimethylglyoxime,
0.412 parts of chain extender,
0.04 parts of crosslinking agent.

3. The functionalized polyurethane according to claim 1, characterized in that:
the self-healing polyurethane elastomer is made from raw materials in the following parts by weight:
1 part of hard segment raw material,
4.5 parts of soft segment raw material,
0.392 parts of dimethylglyoxime,
0.412 parts of chain extender,
0.04 parts of crosslinking agent.

4. The functionalized polyurethane according to claim 1, characterized in that the adamantane-hyaluronic acid composite is made from raw materials in the following parts by weight:
10 parts of hyaluronic acid,
2.3 parts of diepoxy compound,
0.5-5 parts of adamantane.

5. The functionalized polyurethane according to claim 4, characterized in that the adamantane-hyaluronic acid composite is made from raw materials in the following parts by weight:
10 parts of hyaluronic acid,
2.3 parts of diepoxy compound,
5 parts of adamantane.

6. The functionalized polyurethane according to claim 1, characterized in that the raw material for the hard segment is selected from at least one of isophorone diisocyanate and polycaprolactone triol;
and/or, the raw material for the soft segment is selected from at least one of polytetramethylene ether glycol (PTMEG) and polypropylene oxide;
and/or, the chain extender is selected from at least one of 1,4-butanediol and glycerol;
and/or, the crosslinking agent is selected from at least one of aminocyclodextrin and ethylenediamine;
and/or, the diepoxy compound is selected from at least one of 1,4-butanediol diglycidyl ether (BDDE) and 1,6-hexanediol diglycidyl ether.

7. A preparation method for functionalized polyurethane according to claim 1, comprising the following steps:
Step 1: the hard segment raw material reacts with the soft segment raw material to obtain mixture A;
Step 2: mixture A reacts with dimethylglyoxime to obtain mixture B;
Step 3: mixture B reacts with chain extender and crosslinking agent, to obtain self-healing polyurethane elastomer after purification;
Step 4: hyaluronic acid reacts with diepoxide compound to obtain mixture C;
Step 5: mixture C reacts with adamantane to obtain adamantane-hyaluronic acid complex;
Step 6: the adamantane-hyaluronic acid composite is uniformly applied onto the surface of the self-healing polyurethane elastomer to obtain self-lubricating and self-healing polyurethane elastomer materials.

8. The preparation method according to claim 7, characterized in that:
In step 1, the reaction temperature is 60-80° C., and the reaction time is 45 min to 1.5 h;
and/or, in step 2, the reaction temperature is 60-80° C., and the reaction time is 1-2 h;
and/or, in step 3, the reaction temperature is 60-80° C., and the reaction time is 0.5-2 h;
and/or, in step 4, the reaction temperature is 20-50° C., and the reaction time is 6-12 h;
and/or, in step 5, the reaction temperature is 40-80° C., and the reaction time is 12-36 h.

9. Use of functionalized polyurethane according to claim 1 in the preparation of meniscus prostheses.

10. A meniscus prosthesis, characterized in that it is made of functionalized polyurethane according to claim 1.
